(19) United States
(12) Patent Application Publication
(10) Pub. No.: US 2025/0052640 A1
(43) Pub. Date: Feb. 13, 2025

(54) METHOD AND SYSTEM FOR EVALUATING COLLISION TESTING OF BOTTOM OF BATTERY PACK AND DEVICE

(71) Applicants: CATARC New Energy Vehicle Test Center (Tianjin) Co., Ltd., Tianjin (CN); CATARC Co., Ltd., Tianjin (CN)

(72) Inventors: Fang WANG, Tianjin (CN); Pengfei YAN, Tianjin (CN); Tianyi MA, Tianjin (CN); Shiqiang LIU, Tianjin (CN); Lei LIU, Tianjin (CN); Xiaole MA, Tianjin (CN); Yan GAO, Tianjin (CN); Weina WANG, Tianjin (CN); Yue XU, Tianjin (CN); Chenglong JIANG, Tianjin (CN); Liqiong HAN, Tianjin (CN); Ce HAN, Tianjin (CN)

(21) Appl. No.: 18/799,853

(22) Filed: Aug. 9, 2024

(30) Foreign Application Priority Data
Aug. 10, 2023 (CN) 202310999933.5

(51) Int. Cl. G01M 7/08

(52) U.S. Cl. CPC G01M 7/08

(57) ABSTRACT

The present disclosure discloses a method and system for evaluating collision testing of a bottom of a battery pack and a device. The method includes: constructing a three-dimensional battery pack collision simulation model according to material parameters and size parameters of each component at a bottom of a battery pack to be tested; performing collision testing on the battery pack to be tested based on a benchmarking working condition to obtain a measured collision posture; calibrating the three-dimensional battery pack collision simulation model based on the measured collision posture to obtain an optimized three-dimensional battery pack collision simulation model; setting a plurality of standby working conditions, performing collision simulation under any standby working condition by using the optimized three-dimensional battery pack collision simulation model, and recording a simulation result; determining collision safety performance of the battery pack to be tested based on simulation results corresponding to different standby working conditions.

### CROSS-REFERENCE TO RELATED APPLICATION

This patent application claims the benefit and priority of Chinese Patent Application No. 202310999933.5, filed with the China National Intellectual Property Administration on Aug. 10, 2023, the disclosure of which is incorporated by reference herein in its entirety as part of the present application.

### TECHNICAL FIELD

The present disclosure relates to the technical field of battery pack collision testing, and in particular, to a method and system for evaluating collision testing of a bottom of a battery pack and a device.

### BACKGROUND

With the popularization of electric vehicles, requirements for safety performance of battery packs are getting increasingly high. A bottom of a battery pack is an important part of a battery pack structure. When the bottom of the battery pack is subjected to a collision force, the bottom structure of the battery pack is prone to deformation, which leads to the damage of a battery cell in the pack, or even leads to fire and other serious consequences. Therefore, it is particularly important to study methods for evaluating collision testing of a bottom of a battery pack.

Currently, methods for evaluating collision testing of a bottom of a battery pack are mainly a laboratory testing method and a finite element simulation numerical simulation method. The laboratory testing method is visual and reliable, but has a long testing process and a high cost. The numerical simulation method can quickly predict a dynamic response and stress distribution of the battery pack during collision, but accuracy of simulation results is affected by model accuracy, material parameters, and other factors.

### SUMMARY

An objective of the present disclosure is to provide a method and system for evaluating collision testing of a bottom of a battery pack and a device, to improve accuracy and reliability of collision evaluation of the bottom of the battery pack.

To achieve the above objective, the present disclosure provides the following technical solutions.

A method for evaluating collision testing of a bottom of a battery pack includes:
obtaining material parameters and size parameters of each component at a bottom of a battery pack to be tested, and constructing a three-dimensional battery pack collision simulation model according to the material parameters and the size parameters;
setting a benchmarking working condition, and performing collision testing on the battery pack to be tested based on the benchmarking working condition to obtain a measured collision posture;
calibrating the three-dimensional battery pack collision simulation model based on the measured collision posture to obtain an optimized three-dimensional battery pack collision simulation model;
setting a plurality of standby working conditions, performing collision simulation under any standby working condition by using the optimized three-dimensional battery pack collision simulation model, and recording a simulation result, where the simulation result includes a deformation parameter of the battery pack and a stress state parameter at a collision point of the battery pack; and
determining collision safety performance of the battery pack to be tested based on simulation results corresponding to different standby working conditions.

Optionally, working condition data in the benchmarking working condition includes:
material parameters and size parameters of the battery pack to be tested, parameters of a trolley loaded with the battery pack to be tested, collision obstacle parameters, and a collision speed and a collision height of the trolley loaded with the battery pack to be tested.

Optionally, the measured collision posture includes a posture of front wheel jumping and a posture of rear wheel jumping when the trolley loaded with the battery pack to be tested collides with a collision obstacle; and
the calibrating the three-dimensional battery pack collision simulation model based on the measured collision posture to obtain an optimized three-dimensional battery pack collision simulation model specifically includes:
calculating a displacement of a front wheel in a Z direction and a displacement of a rear wheel in the Z direction based on the measured collision posture;
calculating a collision displacement difference according to the displacement of the front wheel in the Z direction and the displacement of the rear wheel in the Z direction; and
constraining, when the collision displacement difference is within a preset threshold range, a Z-direction degree of freedom of the front wheel of the trolley loaded with the battery pack to be tested in the three-dimensional battery pack collision simulation model, where the three-dimensional battery pack collision simulation model after the constraint of the Z-direction degree of freedom of the front wheel is the optimized three-dimensional battery pack collision simulation model.

Optionally, the deformation parameter of the battery pack includes a maximum sag amount of the battery pack and a sag amount of a battery cell, where the maximum sag amount of the battery pack is a Z-direction maximum displacement of recession toward the inside of the battery pack to be tested; and the sag amount of the battery cell is a distance between a bottom surface of the battery cell in the battery pack to be tested and a highest point of a bottom plate of the battery pack; and
the determining collision safety performance of the battery pack to be tested based on simulation results corresponding to different standby working conditions specifically includes:
generating a first collision result if the maximum sag amount of the battery pack is greater than or equal to the sag amount of the battery cell, where the first collision result is used to indicate that the battery pack to be tested is in a dangerous state in terms of the safety performance;
determining a maximum collision stress according to the stress state parameter at the collision point of the battery pack if the maximum sag amount of the battery pack is less than the sag amount of the battery cell;
generating a second collision result if the maximum collision stress is not within a preset stress range, where the second collision result is used to indicate that the battery pack to be tested is in an unsafe state in terms of the safety performance; and
generating a third collision result if the maximum collision stress is within the preset stress range, where the third collision result is used to indicate that the battery pack to be tested is in a safe state in terms of the safety performance.

Optionally, the determining collision safety performance of the battery pack to be tested based on simulation results corresponding to different standby working conditions further includes:
performing, after the second collision result is generated, air tightness verification and insulation performance verification on the battery pack to be tested to obtain a first air tightness and insulation verification result;
generating a fourth collision result if the first air tightness and insulation verification result meets a first preset air tightness and insulation requirement, where the fourth collision result is used to indicate that the battery pack to be tested is in an unsafe state in terms of the safety performance and has good air tightness;
generating a fifth collision result if the first air tightness and insulation verification result does not meet the first preset air tightness and insulation requirement, where the fifth collision result is used to indicate that the battery pack to be tested is in an unsafe state in terms of the safety performance and has poor air tightness;
performing, after the third collision result is generated, air tightness verification and insulation performance verification on the battery pack to be tested to obtain a second air tightness and insulation verification result;
generating a sixth collision result if the second air tightness and insulation verification result meets a second preset air tightness and insulation requirement, where the sixth collision result is used to indicate that the battery pack to be tested is in a safe state in terms of the safety performance and has good air tightness; and
generating a seventh collision result if the second air tightness and insulation verification result does not meet the second preset air tightness and insulation requirement, where the seventh collision result is used to indicate that the battery pack to be tested is in a safe state in terms of the safety performance and has poor air tightness.

To achieve the above objective, the present disclosure further provides the following technical solution:

A system for evaluating collision testing of a bottom of a battery pack includes:
a model construction module, configured to obtain material parameters and size parameters of each component at a bottom of a battery pack to be tested, and construct a three-dimensional battery pack collision simulation model according to the material parameters and the size parameters;
an actual collision test module, configured to set a benchmarking working condition, and perform collision testing on the battery pack to be tested based on the benchmarking working condition to obtain a measured collision posture;
a simulation model optimization module, configured to calibrate the three-dimensional battery pack collision simulation model based on the measured collision posture to obtain an optimized three-dimensional battery pack collision simulation model;
a multi-working condition simulation module, configured to set a plurality of standby working conditions, perform collision simulation under any standby working condition by using the optimized three-dimensional battery pack collision simulation model, and record a simulation result, where the simulation result includes a deformation parameter of the battery pack and a stress state parameter at a collision point of the battery pack; and
a safety performance determining module, configured to determine collision safety performance of the battery pack to be tested based on simulation results corresponding to different standby working conditions.

An electronic device is provided, including a memory and a processor, where the memory is configured to store a computer program, and the processor is configured to run the computer program to enable the electronic device to perform the method for evaluating collision testing of a bottom of a battery pack.

According to specific embodiments of the present disclosure, the present disclosure has the following technical effects:

The present disclosure discloses a method and system for evaluating collision testing of a bottom of a battery pack and a device. A three-dimensional battery pack collision simulation model is constructed according to material parameters and size parameters of each component at a bottom of a battery pack to be tested. The simulation model is constructed by using the above-mentioned collected actual parameters, which can ensure accuracy and reliability of the three-dimensional battery pack collision simulation model. A benchmarking working condition is set, collision testing is performed to obtain a measured collision posture, and then the three-dimensional battery pack collision simulation model is calibrated by using the measured collision posture, which can further improve the accuracy and reliability of the three-dimensional battery pack collision simulation model. Collision simulation under any standby working condition is performed by using an optimized three-dimensional battery pack collision simulation model, and collision safety performance of the battery pack is determined according to simulation results, so that an effective collision situation of the bottom of the battery pack can be obtained, which provides a reference idea for developing a corresponding test system and method. Moreover, through simulation of different standby working conditions, costs of actual testing are reduced, a change trend under different circumstances that fit actual working conditions can be analyzed, and reasonable suggestions can be made accordingly.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a schematic flow chart of a method for evaluating collision testing of a bottom of a battery pack according to the present disclosure;

FIG. 2 is a schematic diagram of a peak change trend of a contact force when collision simulation is performed at different collision speeds in the present disclosure;

FIG. 3 is a schematic diagram of a peak change trend of a contact force when collision simulation is performed at different collision heights in the present disclosure;

FIG. 4 is a schematic diagram of a peak change trend of a collision contact force when collision simulation is performed in different weight ranges in the present disclosure;

FIG. 5 is a schematic diagram of a simulation flow in a specific simulation example according to the present disclosure; and

FIG. 6 is a schematic structural diagram of a system for evaluating collision testing of a bottom of a battery pack according to the present disclosure.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the described embodiments are merely some rather than all of the embodiments of the present disclosure. All other embodiments obtained by those skilled in the art based on the embodiments of the present disclosure without creative efforts shall fall within the protection scope of the present disclosure.

The present disclosure provides a method and system for evaluating collision testing of a bottom of a battery pack and a device, to test safety performance of a bottom structure of the battery pack during collision.

In order to make the above objective, features and advantages of the present disclosure clearer and more comprehensible, the present disclosure will be further described in detail below in combination with accompanying drawings and specific implementations.

### Embodiment 1

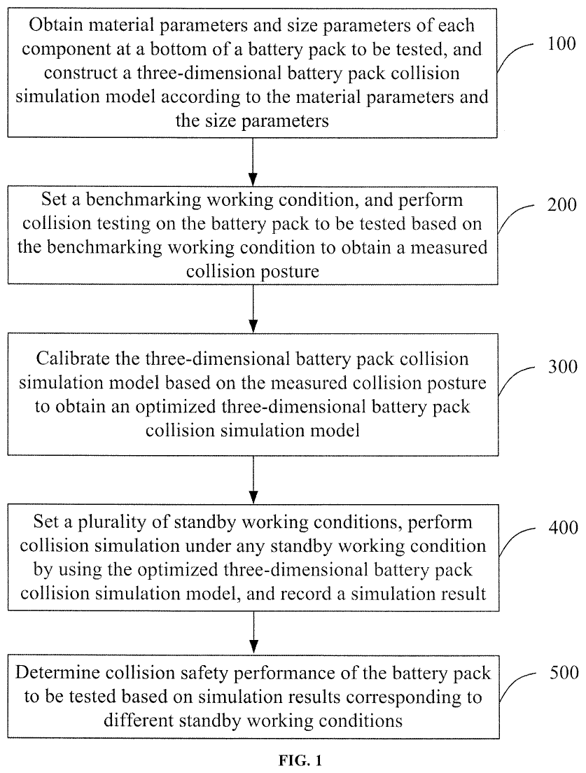

As shown in FIG. 1, the present disclosure provides a method for evaluating collision testing of a bottom of a battery pack, including the following steps.

Step 100: Obtain material parameters and size parameters of each component at a bottom of a battery pack to be tested, and construct a three-dimensional battery pack collision simulation model according to the material parameters and the size parameters.

Generally, the material parameters and the size parameters of each component at the bottom of the battery pack to be tested are collected by means of actual testing, literature research, and the like. The material parameter includes a stress-strain curve, a strength limit, an elastic modulus, a Poisson's ratio and a density of a component material. The size parameter includes a length, a width, a height and a thickness of each component at the bottom of the battery pack to be tested (used for establishing a three-dimensional model).

In SolidWorks software, based on the material parameters and the size parameters, the three-dimensional battery pack collision simulation model is established, and then the three-dimensional battery pack collision simulation model is imported into ANSYS software for collision simulation under different standby working conditions.

Step 200: Set a benchmarking working condition, and perform collision testing on the battery pack to be tested based on the benchmarking working condition to obtain a measured collision posture. Working condition data in the benchmarking working condition includes: material parameters and size parameters of the battery pack to be tested, parameters of a trolley loaded with the battery pack to be tested, collision obstacle parameters, and a collision speed and a collision height of the trolley loaded with the battery pack to be tested.

In a specific test, the benchmarking working condition is set to be consistent with a simulation working condition for simulation analysis in step 300 and step 400. For example, the benchmarking working condition is set to the same battery pack model, the same trolley model loaded with battery packs, the same obstacle model fixed to a ground, the same collision speed, and the same collision height. In addition, the measured collision posture during the testing is captured by using a high-definition camera, and the measured collision posture includes a posture of front wheel jumping and a posture of rear wheel jumping when the trolley loaded with the battery pack to be tested collides with a collision obstacle. The measured collision posture, an acceleration sensor, strain data, an intrusion amount, and the like are used as benchmarking indexes.

Step 300: Calibrate the three-dimensional battery pack collision simulation model based on the measured collision posture to obtain an optimized three-dimensional battery pack collision simulation model.

Step 300 specifically includes the following substeps.
(1): Calculate a displacement of a front wheel in a Z direction and a displacement of a rear wheel in the Z direction based on the measured collision posture.
(2): Calculate a collision displacement difference according to the displacement of the front wheel in the Z direction and the displacement of the rear wheel in the Z direction.
(3): Constrain, when the collision displacement difference is within a preset threshold range, that is, there is a large change trend difference between the displacement of the front wheel in the Z direction and the displacement of the rear wheel in the Z direction, a Z-direction degree of freedom of the front wheel of the trolley loaded with the battery pack to be tested in the three-dimensional battery pack collision simulation model, where the three-dimensional battery pack collision simulation model after the constraint of the Z-direction degree of freedom of the front wheel is the optimized three-dimensional battery pack collision simulation model.

A specific constraint parameter is adjusted as follows: The front wheel Z-direction displacement constraint is set as a front wheel displacement curve of the test trolley captured by the high-definition camera. After the front wheel displacement posture is checked as being consistent with that in the test, the rear wheel posture is naturally consistent.

When the collision displacement difference is not within the preset threshold range, that is, the displacement of the front wheel in the Z direction and the displacement of the rear wheel in the Z direction have the same change trend, effectiveness of the simulation model is indirectly proved, and calibration is not needed.

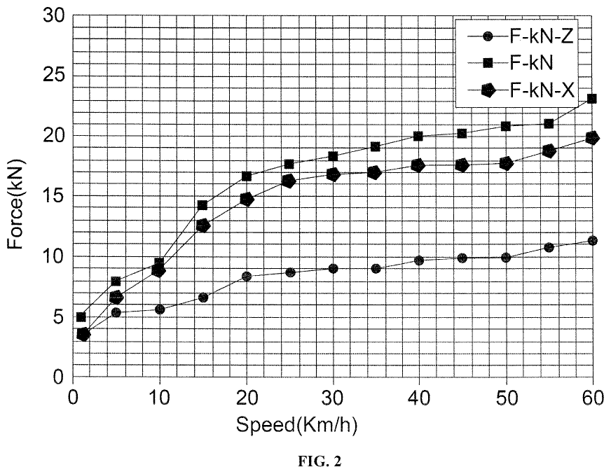

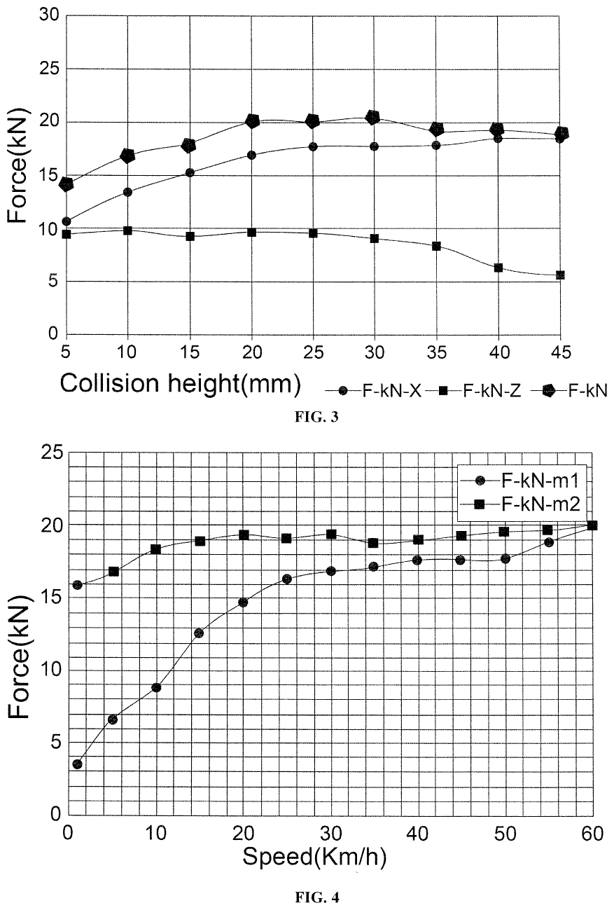

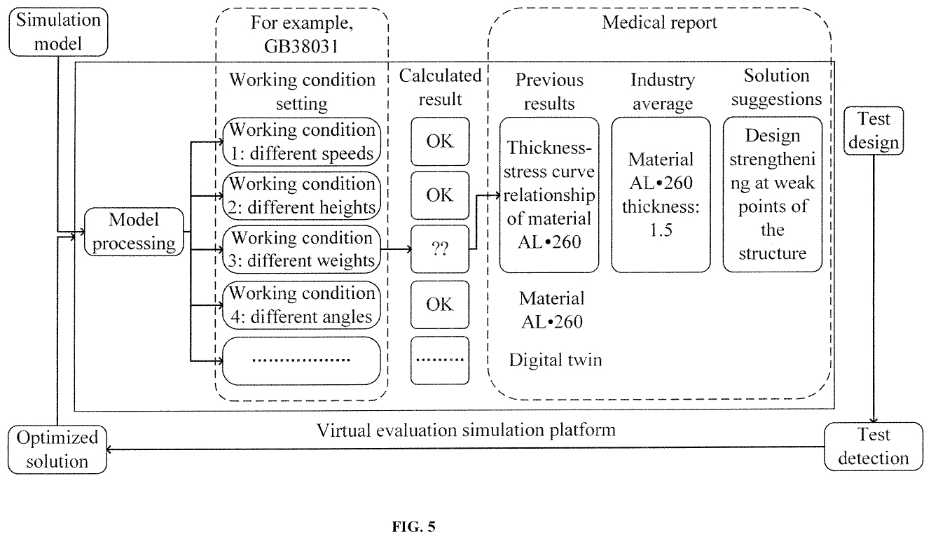

Step 400: Set a plurality of standby working conditions, such as different collision speeds, different collision heights, and different collision weight ranges, perform collision simulation under any standby working condition by using the optimized three-dimensional battery pack collision simulation model, and record a simulation result, as shown in FIGS. 2, 3 and 4, where the simulation result includes a deformation parameter of the battery pack and a stress state parameter at a collision point of the battery pack. The deformation parameter of the battery pack includes a maximum sag amount of the battery pack and a sag amount of a battery cell, where the maximum sag amount of the battery pack is a Z-direction maximum displacement of recession toward the inside of the battery pack to be tested; and the sag amount of the battery cell is a distance between a bottom surface of the battery cell in the battery pack to be tested and a highest point of a bottom plate of the battery pack. FIG. 5 is a schematic diagram of a simulation flow in a specific simulation example. The standby working condition includes working condition 1: different speeds; working condition 2: different heights; working condition 3: different weights; and working condition 4: different angles. There is a risk in calculated results of a simulation output of the working condition 3. Corresponding previous simulation results are counted and drawn into a thickness-stress curve relationship of material AL 260, and corresponding solution suggestions are determined based on an industry average of the material AL 260.

Step 500: Determine collision safety performance of the battery pack to be tested based on simulation results corresponding to different standby working conditions.

Step 500 specifically includes the following substeps.
(1): If the maximum sag amount of the battery pack is greater than or equal to the sag amount of the battery cell, it indicates that the battery cell is squeezed during collision of the bottom of the battery pack, and after the battery cell is damaged, internal short circuit may occur, leading to thermal runaway, fire, and the like. Therefore, if a deformation amount damages the cell in the battery pack, the battery pack to be tested is in a dangerous state in terms of the safety performance. At this time, a first collision result is generated, where the first collision result is used to indicate that the battery pack to be tested is in the dangerous state in terms of the safety performance.
(2): If the maximum sag amount of the battery pack is less than the sag amount of the battery cell, it indicates that the structural deformation amount does not damage the cell, only a certain deformation amount is provided, and a maximum collision stress is determined according to the stress state parameter at the collision point of the battery pack.
(3): If the maximum collision stress is not within a preset stress range (the preset stress range is determined based on the strength limit of the material), it indicates that the battery pack is not seriously broken, and a second collision result is generated, where the second collision result is used to indicate that the battery pack to be tested is in an unsafe state in terms of the safety performance.

After the second collision result is generated, air tightness verification and insulation performance verification are performed on the battery pack to be tested to obtain a first air tightness and insulation verification result.

If the first air tightness and insulation verification result meets a first preset air tightness and insulation requirement, it is proved that the battery pack is really subjected to only certain deformation and certain damage, but remains in an unsafe state and needs to be correspondingly maintained. At this time, a fourth collision result is generated, where the fourth collision result is used to indicate that the battery pack to be tested is in an unsafe state in terms of the safety performance and has good air tightness.

A fifth collision result is generated if the first air tightness and insulation verification result does not meet the first preset air tightness and insulation requirement, where the fifth collision result is used to indicate that the battery pack to be tested is in an unsafe state in terms of the safety performance and has poor air tightness.
(4): If the maximum collision stress is within the preset stress range, it indicates that the structural deformation amount does not damage the cell, and is almost invisible to naked eyes. With reference to the simulation results, a maximum stress of the battery pack to be tested does not exceed a yield limit thereof (a value less than the strength limit), which shows that the battery pack to be tested is not subjected to plastic deformation. At this time, a third collision result is generated, where the third collision result is used to indicate that the battery pack to be tested is in a safe state in terms of the safety performance.

Similarly, after the third collision result is generated, air tightness verification and insulation performance verification are performed on the battery pack to be tested to obtain a second air tightness and insulation verification result.

If the second air tightness and insulation verification result meets a second preset air tightness and insulation requirement, it is proved that the safety state of the battery pack is not changed obviously after the collision. At this time, a sixth collision result is generated, where the sixth collision result is used to indicate that the battery pack to be tested is in a safe state in terms of the safety performance and has good air tightness.

A seventh collision result is generated if the second air tightness and insulation verification result does not meet the second preset air tightness and insulation requirement, where the seventh collision result is used to indicate that the battery pack to be tested is in a safe state in terms of the safety performance and has poor air tightness.

To sum up, according to the present disclosure, the means of testing plus simulation is used to learn from each other and complement each other. The accuracy and reliability of the battery pack bottom collision simulation model can be improved by collecting actual parameters and calibrating the simulation model by the testing means. Through multi-working condition simulation analysis of the simulation results, including different collision speeds, different collision heights, different bottom materials of the battery pack, and other factors that affect the collision safety of the battery pack during the collision, and through post-processing upon the simulation analysis, a collision situation at the bottom of the battery pack can be effectively analyzed, a number of orthogonal matrix tests as an influencing factor is reduced, test costs are greatly reduced, the safety performance of the battery pack structure can be better evaluated, and a basis is provided for the structural optimization design.

### Embodiment 2

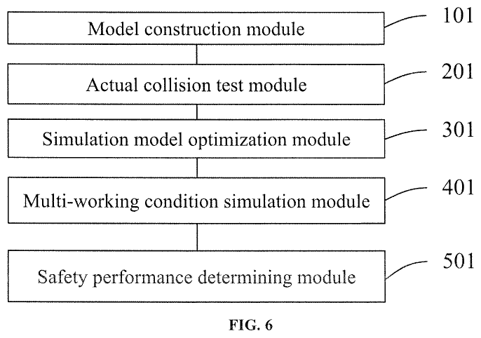

As shown in FIG. 6, in order to achieve the technical solution in Embodiment 1 and achieve corresponding functions and technical effects, this embodiment further provides a system for evaluating collision testing of a bottom of a battery pack, including:
a model construction module 101, configured to obtain material parameters and size parameters of each component at a bottom of a battery pack to be tested, and construct a three-dimensional battery pack collision simulation model according to the material parameters and the size parameters;
an actual collision test module 201, configured to set a benchmarking working condition, and perform collision testing on the battery pack to be tested based on the benchmarking working condition to obtain a measured collision posture;
a simulation model optimization module 301, configured to calibrate the three-dimensional battery pack collision simulation model based on the measured collision posture to obtain an optimized three-dimensional battery pack collision simulation model;
a multi-working condition simulation module 401, configured to set a plurality of standby working conditions, perform collision simulation under any standby working condition by using the optimized three-dimensional battery pack collision simulation model, and record a simulation result, where the simulation result includes a deformation parameter of the battery pack and a stress state parameter at a collision point of the battery pack; and
a safety performance determining module 501, configured to determine collision safety performance of the battery pack to be tested based on simulation results corresponding to different standby working conditions.

### Embodiment 3

This embodiment provides an electronic device, including a memory and a processor, where the memory is configured to store a computer program, and the processor is configured to run the computer program to enable the electronic device to perform the method for evaluating collision testing of a bottom of a battery pack according to Embodiment 1. Optionally, the above-mentioned electronic device may be a server.

In addition, an embodiment of the present disclosure further provides a computer-readable storage medium. The computer-readable storage medium stores a computer program, and when the computer program is executed by a processor, the method for evaluating collision testing of a bottom of a battery pack according to Embodiment 1 is implemented.

Compared with the prior art, the present disclosure further has the following advantages.

According to the present disclosure, through the establishment of the battery pack bottom collision simulation model, combined with actual testing means, the actual parameters are collected and calibrated, so that the accuracy and reliability of the battery pack bottom collision simulation model can be improved. In addition, through the post-processing of the simulation results, the collision of the bottom of the battery pack can be effectively analyzed, and an evaluation method is provided for battery pack bottom collision safety performance.

Embodiments of this description are described in a progressive manner, each embodiment focuses on the difference from other embodiments, and for the same and similar parts between the embodiments, reference may be made to each other. Since the system disclosed in an embodiment corresponds to the method disclosed in an embodiment, the description is relatively simple, and for related contents, reference may be made to the description of the method.

Specific examples are used herein for illustration of principles and implementations of the present disclosure. The descriptions of the above embodiments are merely used for assisting in understanding the method of the present disclosure and its core ideas. In addition, those of ordinary skill in the art can make changes in terms of specific implementations and the scope of application in accordance with the ideas of the present disclosure. In conclusion, the content of this description shall not be construed as limitations to the present disclosure.

What is claimed is:

1. A method for evaluating collision testing of a bottom of a battery pack, comprising:
obtaining material parameters and size parameters of each component at a bottom of a battery pack to be tested, and constructing a three-dimensional battery pack collision simulation model according to the material parameters and the size parameters;
setting a benchmarking working condition, and performing collision testing on the battery pack to be tested based on the benchmarking working condition to obtain a measured collision posture; wherein working condition data in the benchmarking working condition comprises: material parameters and size parameters of the battery pack to be tested, parameters of a trolley loaded with the battery pack to be tested, collision obstacle parameters, and a collision speed and a collision height of the trolley loaded with the battery pack to be tested; and the measured collision posture comprises a posture of front wheel jumping and a posture of rear wheel jumping when the trolley loaded with the battery pack to be tested collides with a collision obstacle;
calibrating the three-dimensional battery pack collision simulation model based on the measured collision posture to obtain an optimized three-dimensional battery pack collision simulation model;
wherein, the calibrating the three-dimensional battery pack collision simulation model based on the measured collision posture to obtain an optimized three-dimensional battery pack collision simulation model specifically comprises: calculating a displacement of a front wheel in a Z direction and a displacement of a rear wheel in the Z direction based on the measured collision posture; calculating a collision displacement difference according to the displacement of the front wheel in the Z direction and the displacement of the rear wheel in the Z direction; and constraining, when the collision displacement difference is within a preset threshold range, a Z-direction degree of freedom of the front wheel of the trolley loaded with the battery pack to be tested in the three-dimensional battery pack collision simulation model, wherein the three-dimensional battery pack collision simulation model after the constraint of the Z-direction degree of freedom of the front wheel is the optimized three-dimensional battery pack collision simulation model;
setting a plurality of standby working conditions, performing collision simulation under any standby working condition by using the optimized three-dimensional battery pack collision simulation model, and recording a simulation result, wherein the simulation result comprises a deformation parameter of the battery pack and a stress state parameter at a collision point of the battery pack; and
determining collision safety performance of the battery pack to be tested based on simulation results corresponding to different standby working conditions.

2. The method for evaluating collision testing of a bottom of a battery pack according to claim 1, wherein the deformation parameter of the battery pack comprises a maximum sag amount of the battery pack and a sag amount of a battery cell, wherein the maximum sag amount of the battery pack is a Z-direction maximum displacement of recession toward the inside of the battery pack to be tested; and the sag amount of the battery cell is a distance between a bottom surface of the battery cell in the battery pack to be tested and a highest point of a bottom plate of the battery pack; and
the determining collision safety performance of the battery pack to be tested based on simulation results corresponding to different standby working conditions specifically comprises:
generating a first collision result if the maximum sag amount of the battery pack is greater than or equal to the sag amount of the battery cell, wherein the first collision result is used to indicate that the battery pack to be tested is in a dangerous state in terms of the safety performance;
determining a maximum collision stress according to the stress state parameter at the collision point of the battery pack if the maximum sag amount of the battery pack is less than the sag amount of the battery cell;
generating a second collision result if the maximum collision stress is not within a preset stress range, wherein the second collision result is used to indicate that the battery pack to be tested is in an unsafe state in terms of the safety performance; and
generating a third collision result if the maximum collision stress is within the preset stress range, wherein the third collision result is used to indicate that the battery pack to be tested is in a safe state in terms of the safety performance.

3. The method for evaluating collision testing of a bottom of a battery pack according to claim 2, wherein the determining collision safety performance of the battery pack to be tested based on simulation results corresponding to different standby working conditions further comprises:
performing, after the second collision result is generated, air tightness verification and insulation performance verification on the battery pack to be tested to obtain a first air tightness and insulation verification result;
generating a fourth collision result if the first air tightness and insulation verification result meets a first preset air tightness and insulation requirement, wherein the fourth collision result is used to indicate that the battery pack to be tested is in an unsafe state in terms of the safety performance and has good air tightness;
generating a fifth collision result if the first air tightness and insulation verification result does not meet the first preset air tightness and insulation requirement, wherein the fifth collision result is used to indicate that the battery pack to be tested is in an unsafe state in terms of the safety performance and has poor air tightness;
performing, after the third collision result is generated, air tightness verification and insulation performance verification on the battery pack to be tested to obtain a second air tightness and insulation verification result;
generating a sixth collision result if the second air tightness and insulation verification result meets a second preset air tightness and insulation requirement, wherein the sixth collision result is used to indicate that the battery pack to be tested is in a safe state in terms of the safety performance and has good air tightness; and
generating a seventh collision result if the second air tightness and insulation verification result does not meet the second preset air tightness and insulation requirement, wherein the seventh collision result is used to indicate that the battery pack to be tested is in a safe state in terms of the safety performance and has poor air tightness.

4. The method for evaluating collision testing of a bottom of a battery pack according to claim 1, wherein the material parameter comprises a stress-strain curve, a strength limit, an elastic modulus, a Poisson's ratio and a density of a component material.

5. A system for evaluating collision testing of a bottom of a battery pack, using the method for evaluating collision testing of a bottom of a battery pack according to claim 1, and comprising:
a model construction module, configured to obtain material parameters and size parameters of each component at a bottom of a battery pack to be tested, and construct a three-dimensional battery pack collision simulation model according to the material parameters and the size parameters;
an actual collision test module, configured to set a benchmarking working condition, and perform collision testing on the battery pack to be tested based on the benchmarking working condition to obtain a measured collision posture;
a simulation model optimization module, configured to calibrate the three-dimensional battery pack collision simulation model based on the measured collision posture to obtain an optimized three-dimensional battery pack collision simulation model;
a multi-working condition simulation module, configured to set a plurality of standby working conditions, perform collision simulation under any standby working condition by using the optimized three-dimensional battery pack collision simulation model, and record a simulation result, wherein the simulation result comprises a deformation parameter of the battery pack and a stress state parameter at a collision point of the battery pack; and
a safety performance determining module, configured to determine collision safety performance of the battery pack to be tested based on simulation results corresponding to different standby working conditions.

6. An electronic device, comprising a memory and a processor, wherein the memory is configured to store a computer program, and the processor is configured to run the computer program to enable the electronic device to perform the method for evaluating collision testing of a bottom of a battery pack according to claim 1.

7. The system for evaluating collision testing of a bottom of a battery pack according to claim 5, wherein the deformation parameter of the battery pack comprises a maximum sag amount of the battery pack and a sag amount of a battery cell, wherein the maximum sag amount of the battery pack is a Z-direction maximum displacement of recession toward the inside of the battery pack to be tested; and the sag amount of the battery cell is a distance between a bottom surface of the battery cell in the battery pack to be tested and a highest point of a bottom plate of the battery pack; and
the determining collision safety performance of the battery pack to be tested based on simulation results corresponding to different standby working conditions specifically comprises:
generating a first collision result if the maximum sag amount of the battery pack is greater than or equal to the sag amount of the battery cell, wherein the first collision result is used to indicate that the battery pack to be tested is in a dangerous state in terms of the safety performance;
determining a maximum collision stress according to the stress state parameter at the collision point of the battery pack if the maximum sag amount of the battery pack is less than the sag amount of the battery cell;
generating a second collision result if the maximum collision stress is not within a preset stress range, wherein the second collision result is used to indicate that the battery pack to be tested is in an unsafe state in terms of the safety performance; and
generating a third collision result if the maximum collision stress is within the preset stress range, wherein the third collision result is used to indicate that the battery pack to be tested is in a safe state in terms of the safety performance.

8. The system for evaluating collision testing of a bottom of a battery pack according to claim 7, wherein the determining collision safety performance of the battery pack to be tested based on simulation results corresponding to different standby working conditions further comprises:
performing, after the second collision result is generated, air tightness verification and insulation performance verification on the battery pack to be tested to obtain a first air tightness and insulation verification result;
generating a fourth collision result if the first air tightness and insulation verification result meets a first preset air tightness and insulation requirement, wherein the fourth collision result is used to indicate that the battery pack to be tested is in an unsafe state in terms of the safety performance and has good air tightness;
generating a fifth collision result if the first air tightness and insulation verification result does not meet the first preset air tightness and insulation requirement, wherein the fifth collision result is used to indicate that the battery pack to be tested is in an unsafe state in terms of the safety performance and has poor air tightness;
performing, after the third collision result is generated, air tightness verification and insulation performance verification on the battery pack to be tested to obtain a second air tightness and insulation verification result;
generating a sixth collision result if the second air tightness and insulation verification result meets a second preset air tightness and insulation requirement, wherein the sixth collision result is used to indicate that the battery pack to be tested is in a safe state in terms of the safety performance and has good air tightness; and
generating a seventh collision result if the second air tightness and insulation verification result does not meet the second preset air tightness and insulation requirement, wherein the seventh collision result is used to indicate that the battery pack to be tested is in a safe state in terms of the safety performance and has poor air tightness.

9. The system for evaluating collision testing of a bottom of a battery pack according to claim 5, wherein the material parameter comprises a stress-strain curve, a strength limit, an elastic modulus, a Poisson's ratio and a density of a component material.

10. The electronic device according to claim 6, wherein the deformation parameter of the battery pack comprises a maximum sag amount of the battery pack and a sag amount of a battery cell, wherein the maximum sag amount of the battery pack is a Z-direction maximum displacement of recession toward the inside of the battery pack to be tested; and the sag amount of the battery cell is a distance between a bottom surface of the battery cell in the battery pack to be tested and a highest point of a bottom plate of the battery pack; and
the determining collision safety performance of the battery pack to be tested based on simulation results corresponding to different standby working conditions specifically comprises:
generating a first collision result if the maximum sag amount of the battery pack is greater than or equal to the sag amount of the battery cell, wherein the first collision result is used to indicate that the battery pack to be tested is in a dangerous state in terms of the safety performance;
determining a maximum collision stress according to the stress state parameter at the collision point of the battery pack if the maximum sag amount of the battery pack is less than the sag amount of the battery cell;
generating a second collision result if the maximum collision stress is not within a preset stress range, wherein the second collision result is used to indicate that the battery pack to be tested is in an unsafe state in terms of the safety performance; and
generating a third collision result if the maximum collision stress is within the preset stress range, wherein the third collision result is used to indicate that the battery pack to be tested is in a safe state in terms of the safety performance.

11. The electronic device according to claim 10, wherein the determining collision safety performance of the battery pack to be tested based on simulation results corresponding to different standby working conditions further comprises:
performing, after the second collision result is generated, air tightness verification and insulation performance verification on the battery pack to be tested to obtain a first air tightness and insulation verification result;
generating a fourth collision result if the first air tightness and insulation verification result meets a first preset air tightness and insulation requirement, wherein the fourth collision result is used to indicate that the battery pack to be tested is in an unsafe state in terms of the safety performance and has good air tightness;
generating a fifth collision result if the first air tightness and insulation verification result does not meet the first preset air tightness and insulation requirement, wherein the fifth collision result is used to indicate that the battery pack to be tested is in an unsafe state in terms of the safety performance and has poor air tightness;
performing, after the third collision result is generated, air tightness verification and insulation performance verification on the battery pack to be tested to obtain a second air tightness and insulation verification result;
generating a sixth collision result if the second air tightness and insulation verification result meets a second preset air tightness and insulation requirement, wherein the sixth collision result is used to indicate that the battery pack to be tested is in a safe state in terms of the safety performance and has good air tightness; and
generating a seventh collision result if the second air tightness and insulation verification result does not meet the second preset air tightness and insulation requirement, wherein the seventh collision result is used to indicate that the battery pack to be tested is in a safe state in terms of the safety performance and has poor air tightness.

12. The electronic device according to claim 6, wherein the material parameter comprises a stress-strain curve, a strength limit, an elastic modulus, a Poisson's ratio and a density of a component material.
